(19) United States
(12) Patent Application Publication
(10) Pub. No.: US 2024/0242847 A1
(43) Pub. Date: Jul. 18, 2024

(54) SYSTEMS AND METHODS FOR MODELING RISK OF TRANSCATHETER VALVE DEPLOYMENT

(71) Applicants: Ohio State Innovation Foundation, Columbus, OH (US); Georgia Tech Research Corporation, Atlanta, GA (US); Emory University, Atlanta, GA (US)

(72) Inventors: Lakshmi Prasad Dasi, Dublin, OH (US); Breandan Andre Butler Yeats, Atlanta, GA (US); Huang Chen, Athens, GA (US); Fateme Esmailie, Atlanta, GA (US); Atefeh Razavi, Atlanta, GA (US); Imran Shah, Newnan, GA (US); Sri Krishna Sivakumar, Dublin, OH (US); Alessandro Veneziani, Decatur, GA (US)

(21) Appl. No.: 18/560,210

(22) PCT Filed: May 11, 2022
(86) PCT No.: PCT/US22/72251

§ 371 Date: Nov. 10, 2023

Related U.S. Application Data
(60) Provisional application No. 63/187,046, filed on May 11, 2021.

(51) Int. Cl. G16H 50/50; G16H 30/40; G16H 50/20; G16H 50/30

(52) U.S. Cl. CPC G16H 50/50; G16H 30/40; G16H 50/20; G16H 50/30

(57) ABSTRACT

A predictive model for classification of tissue rupture risk is generated by providing a computer aided design (CAD) model suitable for simulating a balloon expandable transcatheter heart valve, and computing stress, strain, and/or displacement at the tissue as a function of expansion of the expandable transcatheter heart valve. The computed stress, strain, and/or displacement at the tissue enables determination of low, moderate or high tissue rupture risk as a function of the expansion at time of the expandable transcatheter heart valve deployment into a patient.

### PRIORITY

This application claims priority to U.S. Provisional Patent Application No. 63/187,046, filed May 11, 2021, which is incorporated by reference herein in its entirety.

### BACKGROUND

Aortic root rupture is a rare but fatal complication following TAVR. The likelihood of aortic root rupture has been shown to increase with higher calcium volume and is only known to occur in balloon-expandable transcatheter heart valves (THV). Strategies to prevent rupture include choosing a smaller sized THV than the manufacturers recommendation, underexpanding the THV by lowering the filling volume of the balloon or using a self-expandable THV. However, these strategies can increase the risk of paravalvular leak and create durability issues. Prediction algorithms that can take account for each of these risks can help to better optimize balloon-expandable THV deployment in avoidance of root rupture along with other complications.

Predicting root rupture from routine medical imaging is very difficult due to the complexity and variability of patient anatomy and dynamic nature of the TAVR procedure. Wang et. al. performed a retrospective computational analysis in a single patient who had aortic root rupture measuring the stress in the native tissue following balloon-expandable THV deployment. The location of deployment has also been seen to alter the stress in the aortic root. However, measuring stress has several limitations because of the high variability in patient vessel stiffness and thickness which cannot be measured from routine imaging. Thus, there is value in measuring other parameters that are not as dependent on these variable and unknown patient specific properties. This will also allow a more qualitative risk metric which is based on each of these quantitative measurements. Additionally, computational modeling can capture these parameters at various balloon filling volumes which can help to optimize the procedure based on the aortic root rupture risk parameters.

Prior art methods of patient-specific virtual valve implantation provided only finite element simulations and fail to accurately predict optimal size for valve implant specifically adapted to each patient. Thus the prior art fails to reduce procedural complications and additionally fails to incorporate the impact of surrounding tissue and structures of the aorta

### SUMMARY

An exemplary system and method are disclosed for aortic root rupture prediction following transcatheter aortic valve replacement (TAVR). The exemplary system and method may be applied to reduce aortic root rupture risk through optimizing balloon expansion volume in balloon expandable TAVRs. The exemplary system and method may be employed to measure aortic root rupture risk using stress, strain, and deflection in the aortic root after TAVR. The exemplary system and method may measure these metrics with changes in deployment depth, angle, and off centered for further procedure optimization.

The exemplary system and method may be used for patient preclinical planning for optimization of the procedure. A more suitable procedural approach can be utilized from the increased knowledge gained by the exemplary system and method.

In some aspects, the exemplary system and method employ a classification system that can describe the level of aortic root rupture risk based on the method of quantifying aortic root rupture risk that is more clinically relevant. The exemplary system and method may be used to simulate the mechanical altering of the calcium nodule shape and checking if the aortic root rupture risk is mitigated or not. The exemplary system and method may be used to develop a training database of aortic root rupture cases for the purpose of identifying geometric predictors. The exemplary system and method may use with machine learning, deep learning packages, or other reduced order models that can be trained on these simulations to develop rapidly identifying suspect nodules as well as predicting the geometric and dynamic changes as a function of current and future valve designs. The exemplary system and method may be used for 3D printing from the training database for further experimental validation.

The exemplary system and method may can be used to preoperatively evaluate the risk for aortic root rupture following TAVR and optimize the clinical outcome based on (1) computational simulations, (2) new risk parameters, (3) risk classification, and (4) deployment optimization through computational simulations.

In another aspect, an exemplary system and method is disclosed that can be used to preoperatively evaluate the success of implantation of THV in a surgical heart valve (SHV) with and without expansion from stent fracture, with and without modifications to native heart valve or SHV leaflet tissue and optimize the outcome of the procedure based on computational simulation outcomes.

In another aspect, a predictive model for classification of tissue rupture risk is generated by providing a computer aided design (CAD) model suitable for simulating an expandable transcatheter heart valve, and computing stress, strain, and/or displacement at the tissue as a function of expansion of the expandable transcatheter heart valve. The computed stress, strain, and/or displacement at the tissue enables determination of low, moderate or high tissue rupture risk as a function of the expansion at time of the expandable transcatheter heart valve deployment into a patient.

In another aspect, a method of generating a predictive model for classification of tissue rupture risk includes providing a computer aided design (CAD) model suitable for simulating an expandable transcatheter heart valve, and computing stress, strain, and/or displacement at the tissue as a function of expansion of the expandable transcatheter heart valve. The computed stress, strain, and/or displacement at the tissue enables determination of low, moderate or high tissue rupture risk as a function of the expansion at time of the expandable transcatheter heart valve deployment into a patient.

In another aspect, a patient-specific preoperative model is generated by obtaining patient CT scans, generating an anatomical model, and simulating THV deployment at multiple depths and angles of THV, positions or depth of lacerations for BASILICA/LAMPOON, balloon filling volume and pressures, tissue modifications in the patient anatomy model to determine optimal surgical values for each variable.

In another aspect, a method of preoperatively evaluating the success of a transcatheter heart valve replacement procedure in a patient includes obtaining patient CT scans, generating a patient anatomy model, and simulating THV deployment depth, angle of THV, position or depth of laceration for BASILICA/LAMPOON, balloon volume and pressure, tissue modifications in the patient anatomy model to determine optimal surgical values for each variable.

In another aspect, a method for predictive modeling of transcatheter heart valve deformation using reduced order modeling includes obtaining a library of solutions of selective nodes of a transcatheter heart valve model with a first set of force boundary conditions applied to the selective nodes via finite element simulations. The method also includes predicting deformation of the transcatheter heart valve under a second set of force boundary conditions on the selective nodes via a reduced order model. The second set of force boundary conditions are different from the first set of force boundary conditions.

### BRIEF DESCRIPTION OF THE DRAWINGS

The novel features of the invention are set forth with particularity in the claims. A better understanding of the features and advantages of the present invention will be obtained by reference to the following detailed description that sets forth illustrative aspects, in which the principles of the invention are used, and the accompanying drawings of which:

FIG. 1: Aortic valve segmentation of retrospectively analyzed patient with aortic root rupture occurrence following TAVR (left) and aortic valve segmentation of prospectively analyzed patient without aortic root rupture occurrence following TAVR (right).

FIG. 2: Strain, displacement (mm), and stress (MPa) at a region of calcium protrusion into the native tissue plotted over the balloon filling volume (%) in the retrospectively analyzed patient with aortic root rupture occurrence following TAVR. The patient can be considered moderate risk if the parameters exceed the yellow dotted line and high risk if the parameters exceed the dotted line.

FIG. 3: Strain, displacement (mm), and stress (MPa) at a region of calcium protrusion into the native tissue plotted over the balloon filling volume (%) in the prospectively analyzed patient without aortic root rupture occurrence following TAVR. The patient can be considered moderate risk if the parameters exceed the yellow dotted line and high risk if the parameters exceed the dotted line.

FIG. 4: Surface curvature contours of the patient with root rupture occurrence outlining the change in local surface curvature following the simulated balloon expandable THV.

FIG. 5: Variation of stress with deployment angles between the THV stent and annulus. The stress is seen to increase and decrease in local areas as the stent deployment is rotated with respect to the annulus.

FIG. 6: Example of reduced order modeling showing displacement of a self-expandable stent being represented by 15 nodes.

FIG. 7: Flow chart of the methods to simulate virtual THV implantation in SHV geometry from patient-specific CT scans with SHV fracture and/or tissue modifications.

FIG. 8: Aortic valve segmentation of a patient with failed bioprosthetic aortic valve from pre-procedural CT imaging (left) and simulation of transcatheter heart valve implantation of a prospectively analyzed patient (right).

FIG. 9: Segmentation of SHV in a patient with failed bioprosthetic aortic valve (left) and modification to SHV leaflet geometry for simulation of THV implantation with BASILICA (right).

FIGS. 10A-10C: Aortic valve segmentation of a patient with failed bioprosthetic aortic valve from pre-procedural CT imaging (left) (FIG. 10A), simulation of THV implantation without BASILICA (FIG. 10B), and simulation of THV implantation with BASILICA technique (FIG. 10C) in a patient who was analyzed prospectively and successfully received TAVR with BASILICA based on the results from simulations.

FIGS. 11A-11B: CFD simulation results of the flow into coronaries after idealized TAVR deployment without BASILICA laceration (FIG. 11A) and with BASILICA lacerations (FIG. 11B).

FIGS. 12A-12C: Segmentation of failed bioprosthetic aortic valve from pre-procedural CT imaging (FIG. 12A), virtual simulation of THV implantation inside failed SHV without SHV fracture (FIG. 12B), and high-pressure balloon expansion to simulate fracture of SHV with implanted THV (FIG. 12C).

FIG. 13: Difference in the THV stent diameters measured at outflow, waist and inflow locations of THV between virtual predictive simulation of THV implantation in fractured SHV and in-vitro experiments of 20 mm THV in 19 mm SHV and 23 mm THV in 21 mm SHV with fracture.

FIGS. 14A-14C: Patient segmentation including the mitral anterior and posterior cusps, left ventricle, left atrium, LVOT, and calcium nodules (FIG. 14A), anterior cusp and a large calcium nodule at its base (FIG. 14B), and simulation of LAMPOON procedure cut along the anterior leaflet to the calcium nodule (FIG. 14C).

FIGS. 15A-15D: Simulation results of the SAPIEN 26 mm without LAMPOON (FIG. 15A), with LAMPOON (FIG. 15B), SAPIEN 29 mm without LAMPOON (FIG. 15C), and with LAMPOON (FIG. 15D).

FIGS. 16A-16C: Neo-LVOT area assessment. Cross-sectional view of the pre-procedural CT scan with the simulated SAPIEN 3 29 mm results overlaid detailing the ventricle and atrium in purple, anterior cusp in orange, calcium in blue and stent in pink, the simplified stent implantation is also overlaid in teal (FIG. 16A). Three-dimensional rendering detailing the significance of the anterior cusp and LVOT obstruction (FIG. 16B). Neo-LVOT area comparison between the simulated and simplified deployment methods with the simulated method resulting in a much smaller area (73 mm² compared to 282 mm²) (FIG. 16C).

FIGS. 17A-17B: CFD results detailing velocity contours through the neo-LVOT. High peak velocities (6.1 m/s) without the LAMPOON procedure (FIG. 17A) and lowered peak velocities (5.0 m/s) with the LAMPOON procedure (FIG. 17B).

FIG. 18 shows summary of the reduced order modeling (ROM) framework, which can be split into an offline phase, where the computationally expensive simulations are offloaded, followed by a prompt recycling of the trained data set in the online phase for a new set of parameters.

FIGS. 19A-19B show idealized model of the Evolut R stent frame, from which three planes P1, P2, and P3 are defined along which all force-pair boundary conditions are applied (FIG. 19A). Sample force pair boundary condition applied between two nodes of the stent (FIG. 19B).

FIGS. 20A-20B show Eigenvalue decay of stent deformation following POD implementation (FIG. 20A). Retained energy calculated for each number N of reduced bases captured (FIG. 20B).

FIGS. 21A-21B show comparison between the stent deformation from the ROM simulation (FIG. 21A) and the FOM finite element simulation (FIG. 21B) after simulating stent crimping.

FIGS. 22A-22B show comparison between stent deformation from ROM simulations and the FOM finite element simulation for stent expansion.

FIG. 23 shows an illustrative computer architecture for a computer system 200 capable of executing the software components that can use the output of the exemplary method described herein.

### DETAILED DESCRIPTION

### Example Method

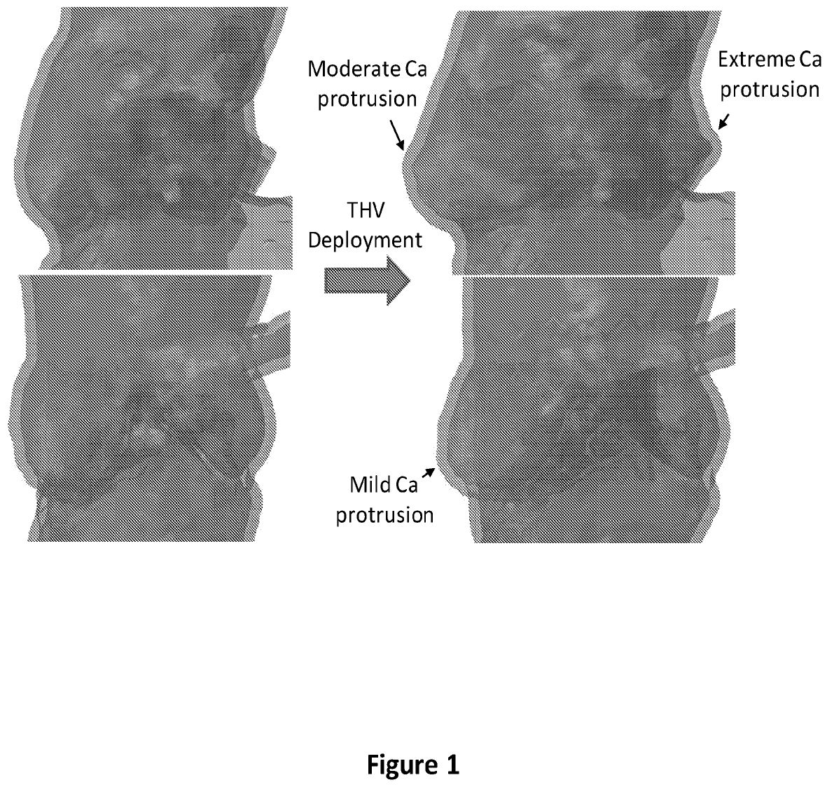

To demonstrate tissue rupture prediction feasibility two patient pre-operative computed tomography scans were taken. One was analyzed retrospectively, and the other was analyzed before the TAVR procedure. The retrospective patient did have root rupture occurrence following TAVR and the other did not. Both aortic valves were segmented including leaflets and calcium and the balloon-expandable THV was deployed in both anatomies (FIG. 1). Large protrusions are seen in the patient who did have aortic root rupture caused by the calcium displacing into the native tissue (FIG. 1). These protrusions are very difficult to predict from just looking at routine imaging. For example, clinical studies have observed calcium volume to increased risk of aortic root rupture however, in this case the calcium volume was significantly higher in the patient who did not have root rupture (1164 mm³ and 1701 mm³ calcium volume with and without root rupture respectively).

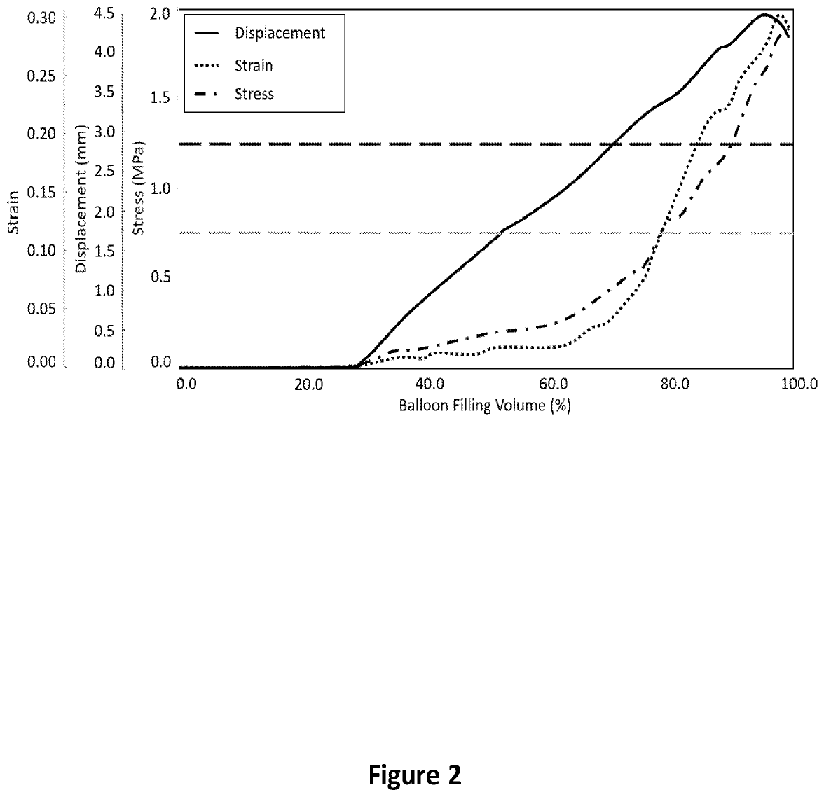

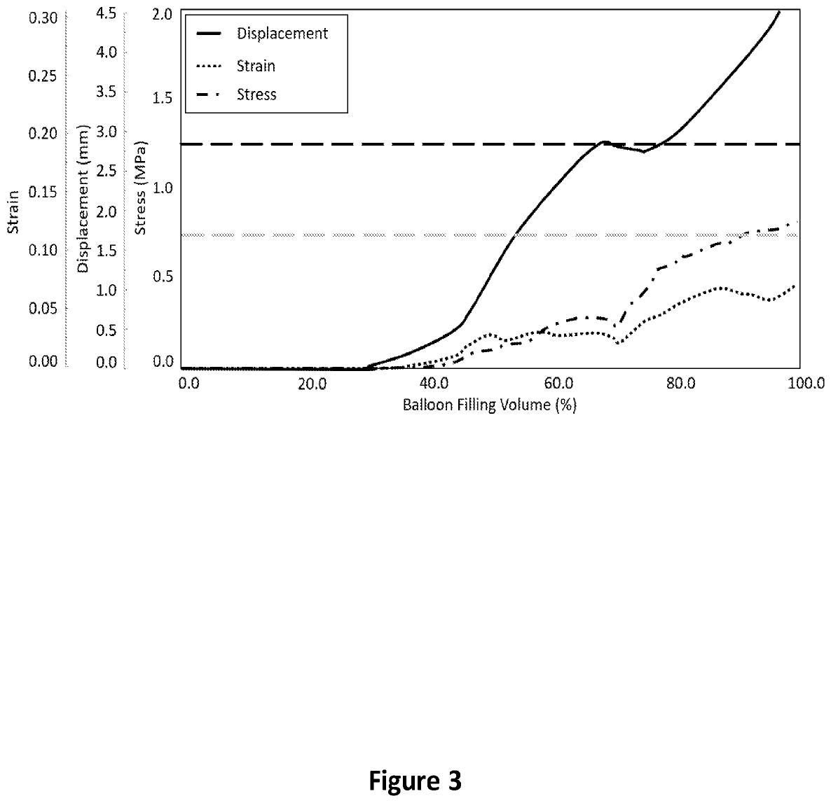

The level of protrusion in these regions can be further quantified using several parameters including stress, strain, and displacement. These parameters are plotted with respect to the balloon filling volume for the patient with root rupture (FIG. 2) and without root rupture (FIG. 3).

The root rupture patient is seen to have higher rupture risk based on stress and strain at the localized protrusion region. These values can also be characterized by implementing risk cutoff points for low, moderate, and high risk for aortic root rupture. Example cutoff values are shown in Table 1.

TABLE 1

| | Stress (MPa) | Strain | Displacement (mm) |
|---|---|---|---|
| Low | <0.75 | <0.12 | <1.7 |
| Moderate | 0.7-1.25 | 0.12-0.18 | 1.7-2.8 |
| High | >1.25 | >0.18 | >2.8 |

Table 1 shows aortic root rupture risk classification of low, moderate, and high risk based on computational measurements of stress (MPa), strain, and displacement (mm) in localized regions of calcium protrusion following balloon-expandable THV simulated deployment.

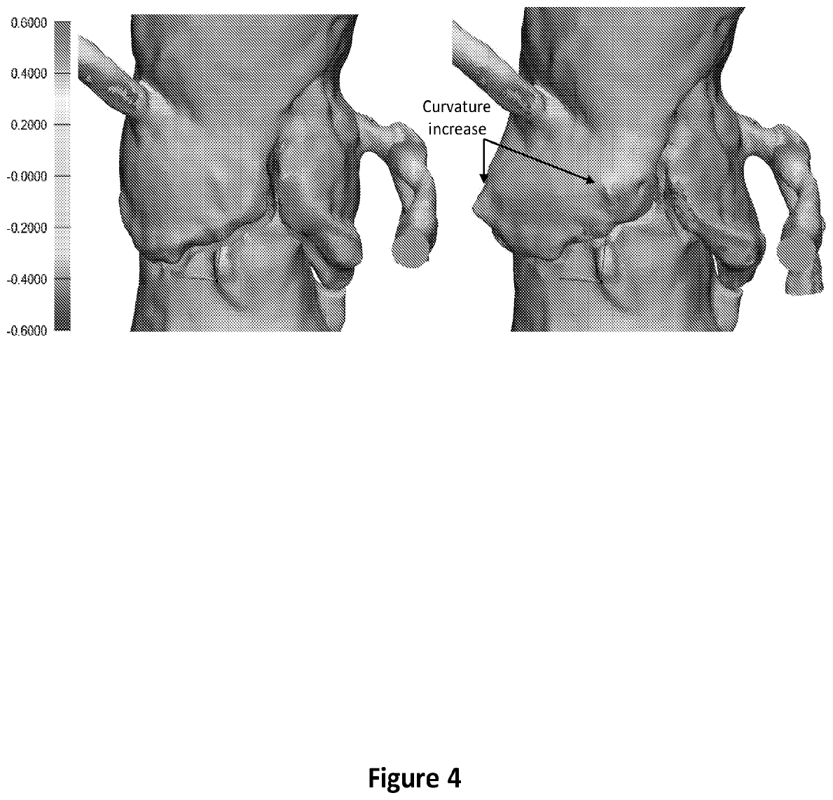

This risk of aortic root rupture has been seen to change with balloon filling volume. If this analysis is performed before the procedure it can aid in clinical planning by giving the clinician an optimized filling volume that they can use during the procedure. This is demonstrated by these two patients where the prospective patient underwent TAVR with a lowered balloon filling volume and did not experience root rupture. Additionally, this type of risk assessment provides a great deal of additional information that cannot be known from only looking at the CT scan as seen from the differences of normal aortic root rupture risk assessment using only calcium volume where the patient who did have root rupture had significantly less calcium volume however, had much higher stress and strain. Further analysis such as altering the deployment depth, angle, and position can be easily implemented for further optimization. This analysis can also be easily performed for other valve replacement and stent deployment therapies. Additional metrics such as local surface curvature changes can also be measured and correlated to aortic root rupture risk (FIG. 4).

FIG. 4 shows surface curvature contours of the patient with root rupture occurrence outlining the change in local surface curvature following the simulated balloon-expandable THV.

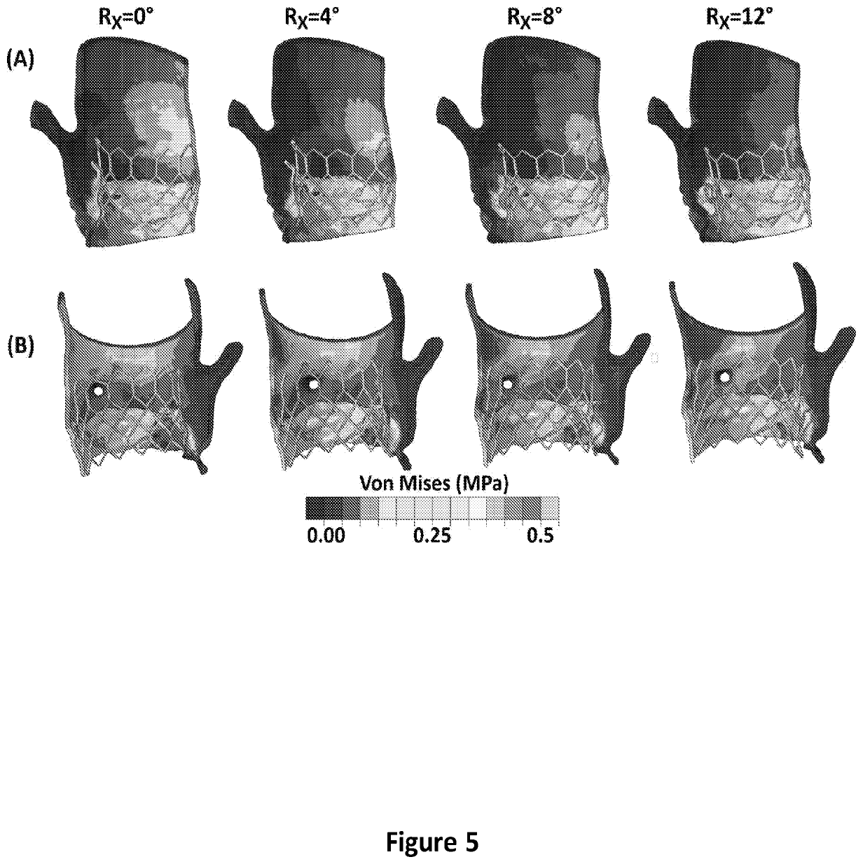

Further analysis such as altering the deployment depth, angle, and position can be easily implemented for further optimization (FIG. 5). FIG. 5 shows stress variation with various deployment angles between the stent and annulus.

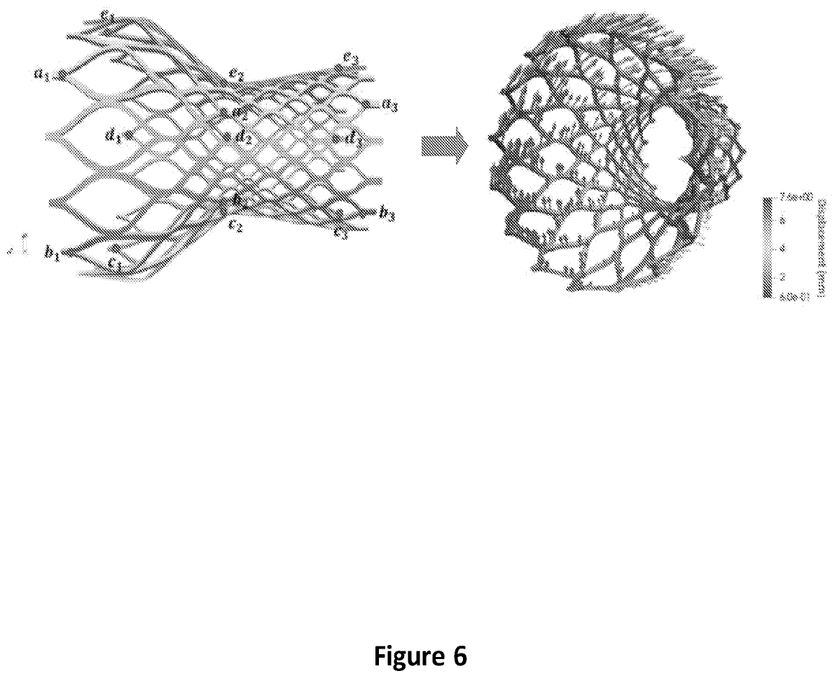

In some aspects, a training database of patients with and without aortic root rupture may be implemented. The database may take patient anatomical parameters and correlate them to the incidence of aortic root rupture for better identifying high risk patients. Additionally, machine learning algorithms and reduced order models may be implemented to rapidly identify suspect calcium nodules and rapidly predict the geometric and dynamic changes of the anatomy for all future valve designs. Reduced order modeling proof of concept is shown in FIG. 6.

Additionally, developing a training database will allow for the development of a threshold for balloon pressure which has high risk of rupture. Balloon pressure can be measured during the procedure using an attached pressure gauge. Locking mechanisms can be implemented which automatically stop balloon filling when the balloon pressure exceeds the maximum threshold preventing the operator from accidentally exceeding the high risk threshold. This can be extended to computationally finding the optimal device placement with regard to deployment depth, position, and balloon volume then exactly performing this deployment in the patient using precise robotic methods which is able to be programmed and have complete control of the deployment apparatus.

Finally, 3D printing from the training database may be used for further experimental validation.

Method to Simulate Stent Deployment Inside Stented Tissue Valve or Native Heart Valve with Geometry Modifications

Aortic valve stenosis is the most common cause for heart valve replacement. The treatment for aortic valvular disease is surgical valve replacement where a mechanical or bioprosthetic valve is used to replace a malfunctioning native aortic valve. Patients who are at risk of mortality from surgical valve replacement due to comorbidities are now able to be treated with transcatheter aortic valve replacement (TAVR) which is a minimally invasive alternative way to replace a diseased valve. TAVR has been shown to be effective in not only replacement of native aortic valve but also a failed bioprosthetic aortic valve in patients who are at high risk of mortality for redo-surgery.

Coronary occlusion or obstruction (CO) is a rare procedural complication during TAVR. It is the partial or complete obstruction of flow of blood in the coronary arteries starting from the aorta that supply oxygenated blood to the heart muscles. Unlike open heart surgical aortic valve replacement, the pre-existing aortic valve leaflets are not removed during TAVR which results in cases with high chances of one or both coronaries being obstructed due to the deployment of the TAV device. Patients undergoing TAVR in failed bioprosthetic aortic valve are at increased risk of coronary obstruction due to leaflets of the bioprosthetic valve forming a closed cylinder after implantation of THV that can stop coronary perfusion. Bioprosthetic or native aortic scallop intentional laceration to prevent iatrogenic coronary artery obstruction (BASILICA) is a novel technique developed to mitigate the risk of coronary obstruction in patients undergoing TAVR. However, it is difficult to visualize how the SHV leaflets end up after BASILICA and implantation of the THV from conventional clinical imaging techniques. The angle of laceration on the leaflet cusp, depth of laceration, implantation angle, depth, and size of THV can all play a role in the final position taken by the SHV leaflets and decide whether the risk of obstruction has been eliminated. Computational patient specific modeling of THV implantation in failed SHVs can help optimize the technique and improve outcomes of TAVR while preventing life threatening complications.

Bioprosthetic valve fracture (BVF) has been demonstrated to increase the internal dimensions of failed surgical valves, allowing for optimal expansion of the THV inside the failed surgical valve with reduced pressure gradient and improved effective orifice area (EOA). However, BVF may increase the risk of complications such as coronary obstruction and root rupture. Predicting the success of BVF from clinical imaging methods such as echocardiography and computed tomography (CT) is difficult due to the complexity involved in the geometry and the interaction of the SHV and THV. Patient specific computational modeling of BVF can not only assess the possibility of achieving the hemodynamic success intended with BVF but also identify the risk of adverse outcomes such as coronary obstruction or root rupture.

Patients with a failed native or bioprosthetic heart valve in the mitral position who are considered high risk for surgical valve replacement undergo transcatheter mitral valve replacement (TMVR). Left ventricular outflow tract (LVOT) obstruction is a potentially fatal complication after TMVR caused by the protrusion of the native anterior mitral leaflet in the LVOT. LVOT obstruction can be characterized by elevated outflow velocities and increased pressure gradient. Patients who are at high risk of obstruction of LVOT undergo anterior mitral leaflet laceration (LAMPOON technique) to mitigate the risk of obstruction, enabling additional blood flow into the LVOT. Accurate modeling of the risk of LVOT obstruction is critical for appropriately selecting patients who can safely undergo TMVR. Currently, the only way of estimating LVOT obstruction is by using CT imaging which does not account for the displacement of the anterior mitral leaflet and calcification after THV implantation. Computational modeling of TMVR implantation and subsequent investigation of hemodynamics with and without LAMPOON can be used to optimize the technique and achieve excellent clinical outcomes.

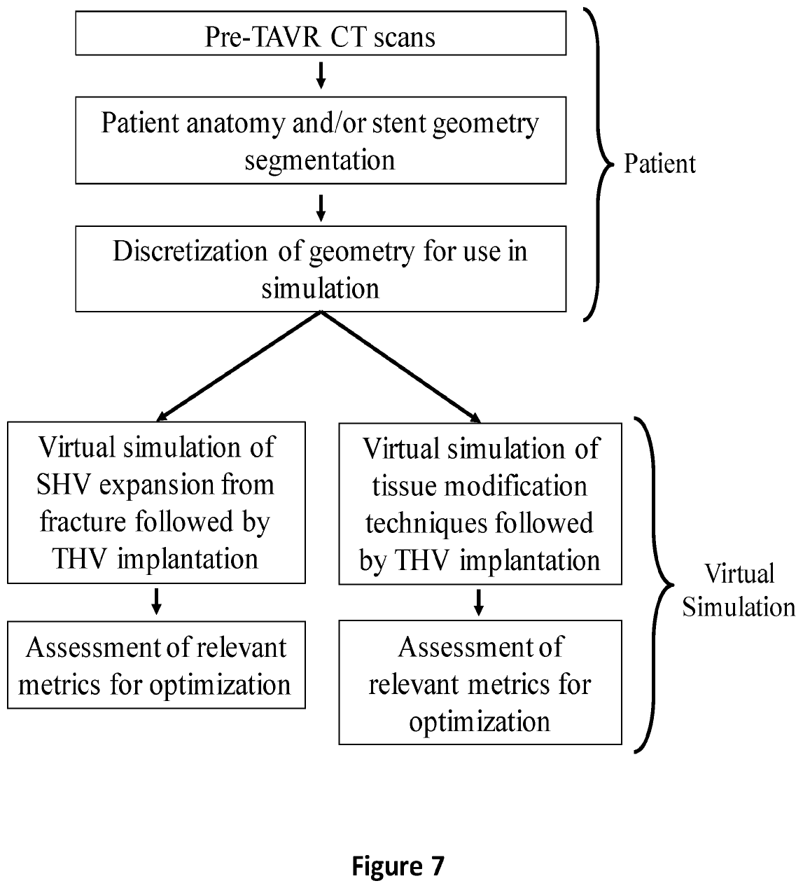

The exemplary method and system can be used to preoperatively evaluate the success of implantation of THV in SHV with and without expansion from stent fracture and optimize the clinical outcome based on computational simulations. FIG. 7 shows an example method to simulate virtual THV implantation in SHV geometry from patient-specific CT scan.

The exemplary methods described herein employ computational modeling techniques to provide a great deal of additional information that is generally not available to a clinician from a CT scan. Techniques described above that are used during THV in SHV or THV in native implantation can be virtually simulated and assessed using metrics such as coronary flow velocities to determine their chances of success in the patient using patient specific modeling. Further analysis such as altering the deployment depth, angle of THV, position, depth of laceration for BASILICA/LAMPOON, balloon volume & pressure can be easily implemented for further optimization.

The exemplary methods described herein can be used to develop a predictive model for obtaining the pressure gradient across THV after implantation with and without geometry modifications. Simulation of THV deployment by adding THV leaflets to the THV stent geometry can be used to obtain the geometric orifice area of the transcatheter heart valve after implantation of the THV in SHV with and without SHV fracture and/or THV in native heart valves with tissue modifications to SHV or native heart valves. Further analysis using preoperative echocardiography imaging data can give predictions of pressure gradient across the heart valve, that dictates the success of valve replacement procedure, from only virtual deployment of THV in patient specific anatomy using FEA without the need for expensive CFD simulations.

The exemplary method could also implement a training database of patients with and without successful THV in SHV implantation with techniques of BVF, BASILICA or LAMPOON. Additionally, artificial intelligence and or machine learning algorithms and reduced order models may be implemented to develop a training database of virtual simulations of THV deployment with tissue and geometry modifications for rapid identification of outcomes of THV in SHV as well as predicting the geometric and dynamic changes as a function of current and future valve designs. Finally, 3D printing from the training database will be used for further experimental validation.

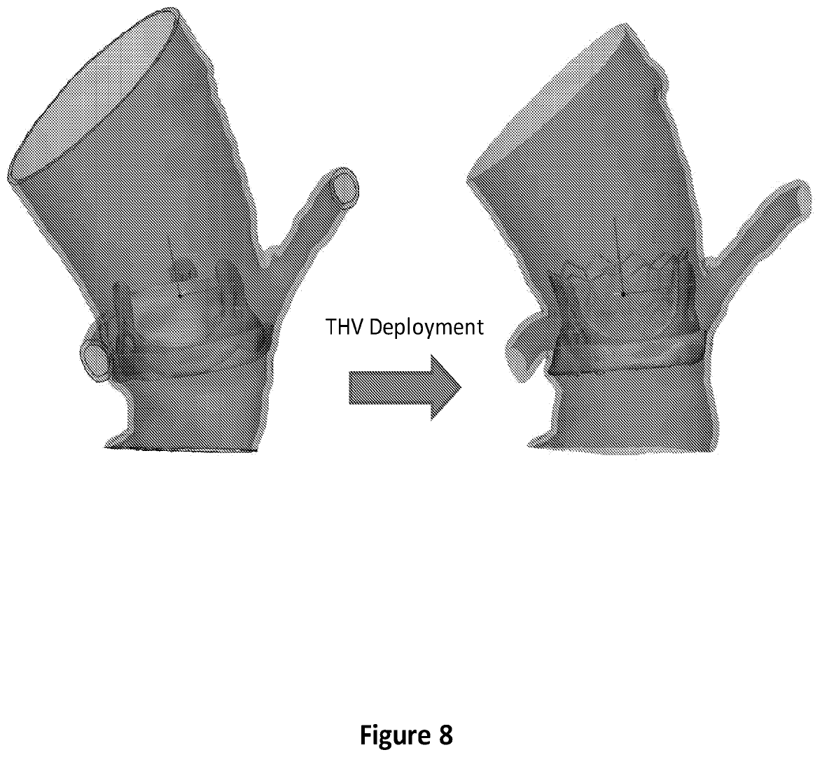

To demonstrate the feasibility of modeling THV implantation in failed SHV with BASILICA, pre-procedural CT images of a 74-year-old female patient who had a failed 21 mm bioprosthetic aortic valve who was in consideration for valve-in-valve TAVR was obtained prospectively. Patient specific virtual models of the aortic root, bioprosthetic valve stent and leaflets were reconstructed from CT images and discretized with 3-D tetrahedral elements (FIG. 8).

FIG. 8 shows an aortic valve segmentation of a patient with failed bioprosthetic aortic valve.

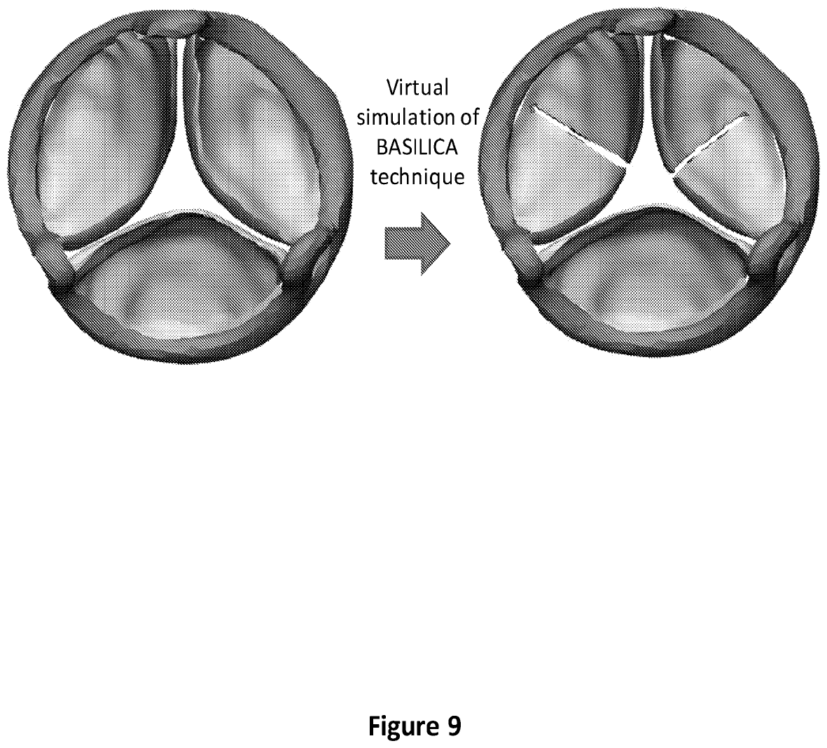

Material models for the root, leaflets and stent were derived from literature. To emulate the BASILICA technique, another model of the patient geometry was used where a cut was created in the geometry of the leaflet model from the tip of the leaflet cusp to the base of the leaflet in both the left and right coronary leaflet cusps of the SHV (FIG. 9).

FIG. 9 shows an aortic valve segmentation of a patient with failed bioprosthetic aortic valve and modifications to the geometry for simulation of THV implantation with tissue modifications.

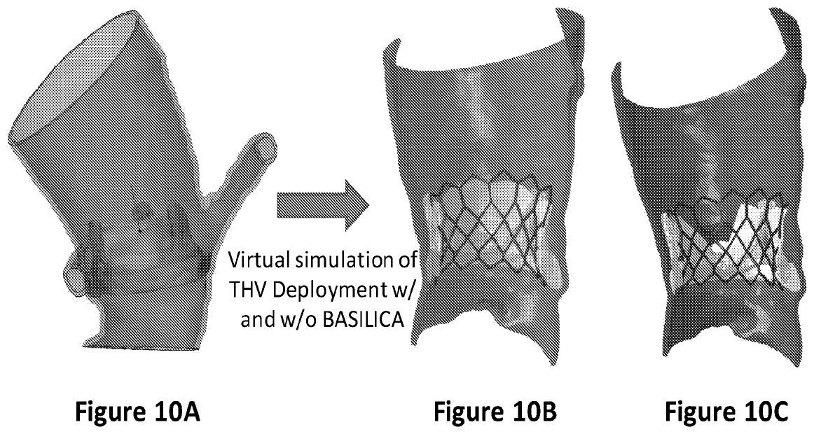

Virtual THV implantation was simulated in both models by using finite element methods. DLC/d is a risk factor that is the ratio of the distance of the leaflet from the coronary ostium to the diameter of the coronary ostium. By assessment of the risk of coronary obstruction after THV implantation in SHV using the risk factor DLC/d, it was shown that the DLC/d was 0.32 and 0.27 for the left and right coronary arteries suggesting very high risk of coronary obstruction. However, in the case with BASILICA technique simulated, the obstruction in both coronaries were completely removed and no leaflet material of the SHV was found to be in front of either coronary artery (FIGS. 10A-10C).

The patient successfully received TAVR with bilateral BASILICA based on the results from modeling, showing the capabilities of modeling in predicting success of not only THV in SHV but also the tissue modification techniques used to prevent adverse outcomes in such procedures.

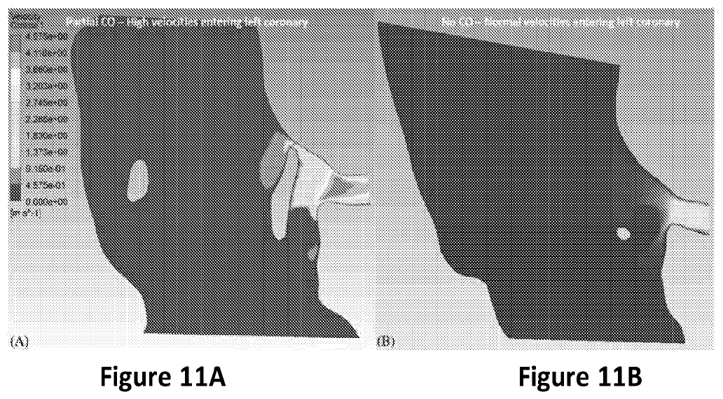

FIGS. 11A-11B show simulation results of the flow velocities into coronaries in a computational THV implantation with and without BASILICA.

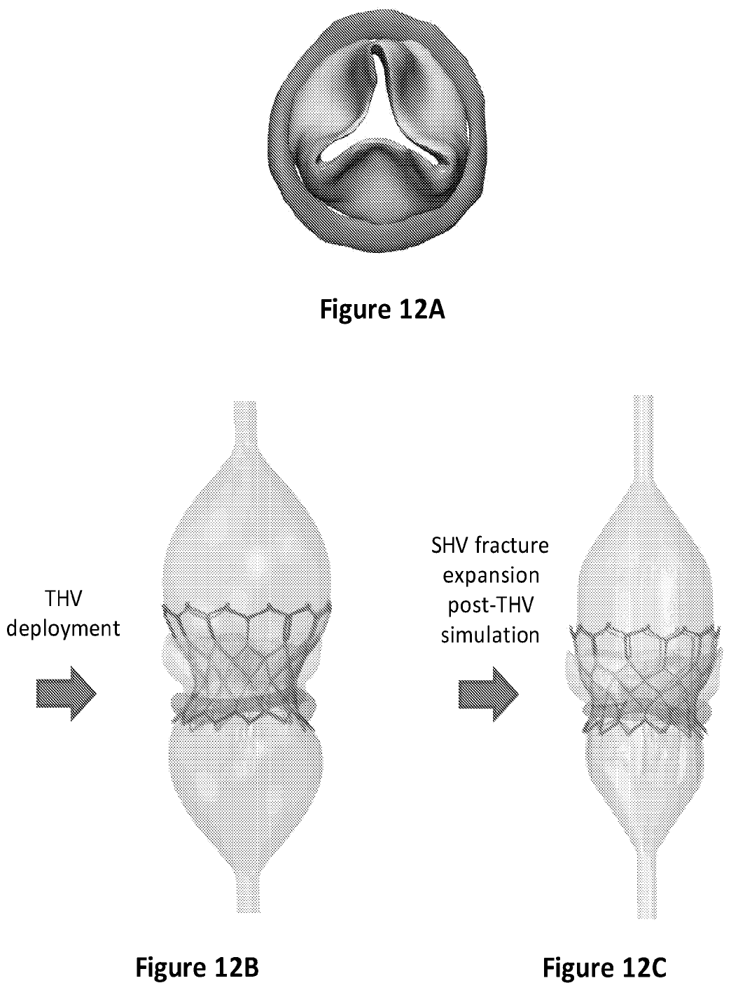

To demonstrate the feasibility of modeling valve fracture and subsequent modeling of transcatheter aortic valve virtual implantation, pre-operative computed tomography scans were obtained of patients with failed bioprosthetic aortic valves that are compatible with BVF. Patient specific geometry models of the aortic root, bioprosthetic valve were constructed from CT images using segmentation techniques and discretized with three dimensional tetrahedral elements. The geometry of the THVs were generated and discretized with hexahedral elements. Material models used for aortic root, bioprosthetic valve and THV were derived from literature. The geometry of the THVs were generated and discretized with hexahedral elements. Virtual implantation of THV in failed SHV was simulated using finite element techniques (FIG. 12B). After the implantation of THV, a high-pressure balloon was used to simulate fracture of the SHV and expand the area available for the THV inside the SHV (FIG. 12C).

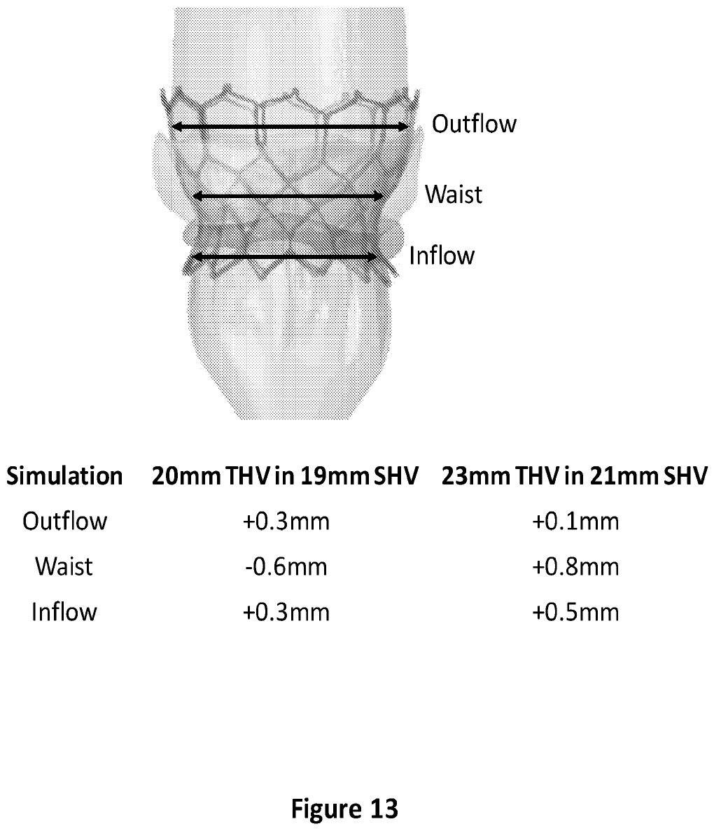

Comparison of the diameters of the THV after virtual simulation of THV implantation measured at outflow, waist and inflow regions showed great agreement with measurements from in-vitro simulation of the same procedure (FIG. 13).

The methods used here demonstrate that BVF can be predicted accurately in virtual simulation of THV in SHV and further assessment of metrics can be used in optimization of BVF for best procedural outcomes.

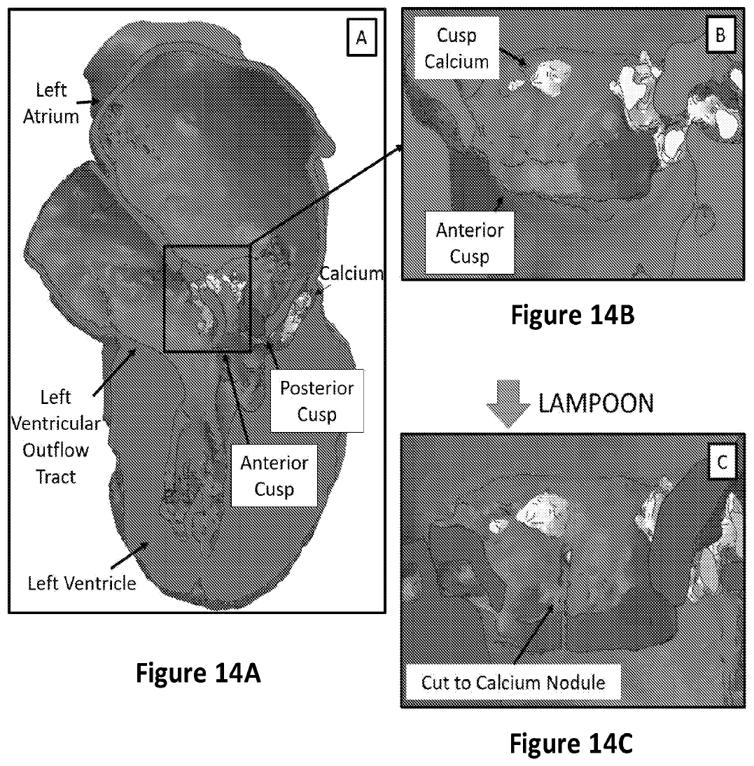

A 60-year-old female with severe mitral annular calcification (MAC) who was considered for THV implantation in the mitral position was considered. Preprocedural cardiac computed tomography (CT) was performed and obtained for analysis under TRB approved protocols. The mitral valve, left ventricle, left atrium, aortic root, and calcium were segmented (FIGS. 14A-14B). The geometry of the THVs were generated and discretized with hexahedral elements. Material models were used from literature. The LAMPOON procedure was simulated by removing a line of elements from the middle free edge along the belly of the cusp to the large calcium nodule at the base (FIG. 14C).

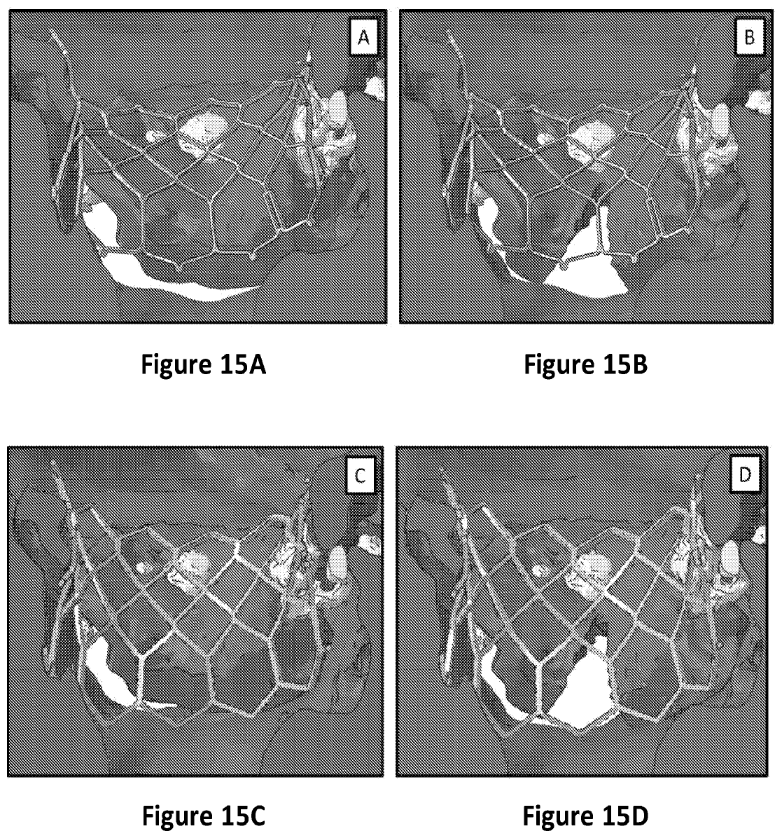

Both stents were deployed with and without LAMPOON, the neo-LVOT area was then observed in all four cases (FIGS. 15A-15D).

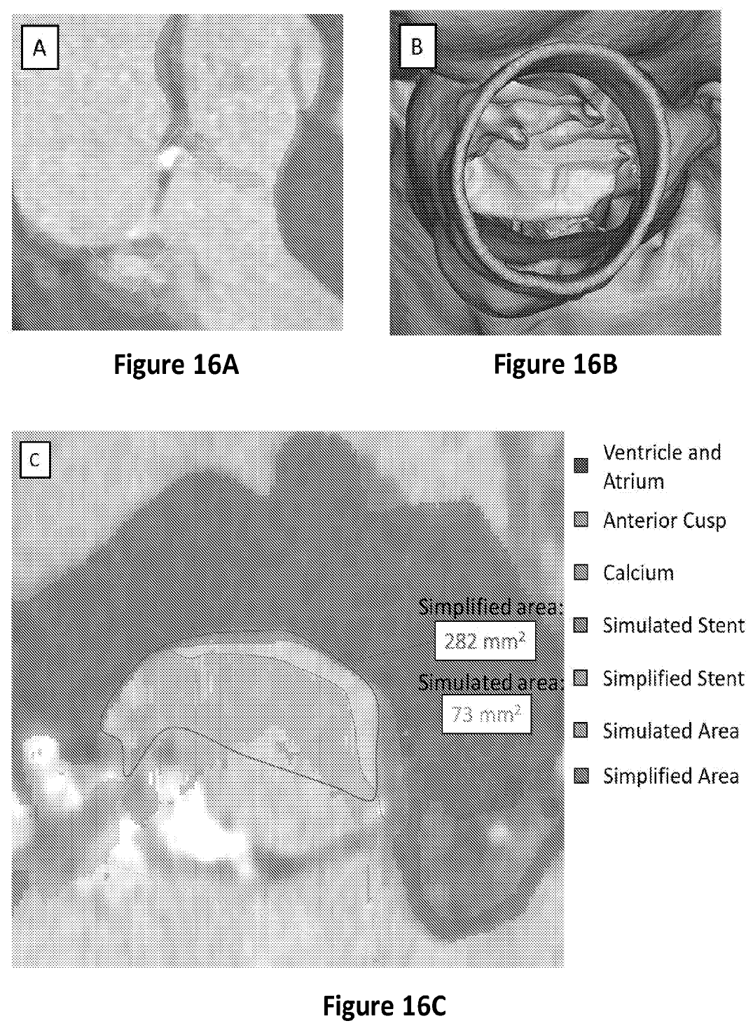

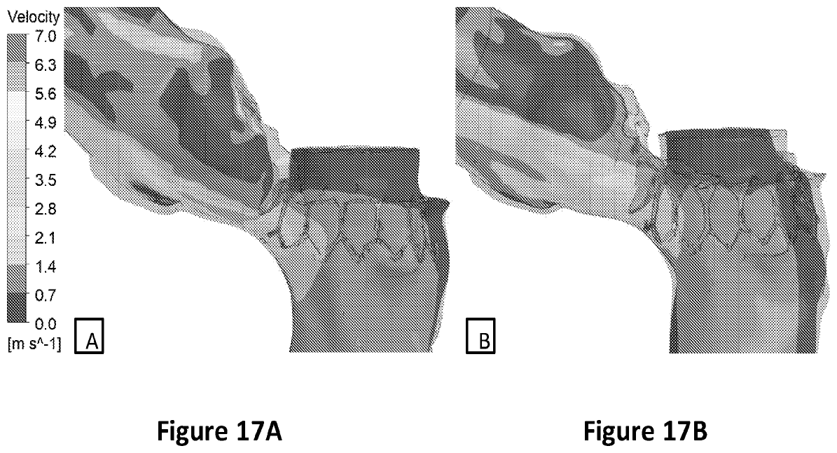

As shown in FIGS. 16A-16C, the neo-LVOT area was estimated by creating a spline through the neo-LVOT and measuring the minimum area. This was compared with the simplified method of neo-LVOT obstruction prediction which consists of overlaying a cylinder in the mitral position. For the 20 mm THV case, a large difference in area of the neo-LVOT (73 mm² compared to 282 mm²) was observed between the complex finite elemental simulation and the simplified stent deployment, the area was increased to 104 mm² after LAMPOON however, it is still ˜2.7 times smaller than the simplified deployment method. This is likely due to the simplified deployment not factoring the structural deformation of the anterior cusp, incomplete splaying of the cusp after LAMPOON, and irregular THV expansion caused from severe annular and cusp calcification. The effect of LAMPOON was further investigated through the use of computation fluid dynamics (CFD) on the post simulation results following methods detailed in a previous study. The 29 mm THV implantation with and without LAMPOON cases were analyzed. As shown in FIGS. 17A-17B, without LAMPOON the velocities through the neo-LVOT were higher.

The pressure gradient between the left ventricle and the sinotubular junction without LAMPOON and with LAMPOON were 156 mmHg and 86 mmHg respectively. These high pressure gradients are consistent with reported clinical measurements in patients with neo-LVOT areas <100 mm2. In this case LAMPOON was not sufficient in preventing LVOT obstruction (pressure gradient>10 mmHg) and there was a significant discrepancy in the neo-L VOT area between the simulated and simplified models with the simulated model predicting a high risk of LVOT obstruction and the simplified model predicting a low risk of LVOT obstruction.

High accuracy modeling can be used in this manner to better predict neo-LVOT area and analyze the patient hemodynamics prior to the clinical procedure. Validation of this methodology through its use in more patients with post operation CT and echocardiography will be extremely valuable. CFD can implement the flow across the cardiac cycle and the geometry of the native or bioprosthetic aortic valve to give temporal information on how LVOT obstruction effects the pressure gradient across the aortic valve. This will give information on what levels of LVOT obstruction may be acceptable in high-surgical risk patients which only have transcatheter treatment options.

Through an iterative technique the risk of complications can be optimized. This can be performed by making changes in the device position, balloon volume, location and length of the laceration for BASILICA/LAMPOON, etc. The most optimal deployment can then be performed.

This technique can also be used in testing future device designs. The efficacy of geometry modification techniques may not be consistent between all devices which are deployed. Additionally, the new device deployment simulation can be performed with and without native geometry modification and compared to the current standard devices.

The exemplary methods described herein can be used to identify optimal device design of future THVs for preventing complications such as tissue rupture, coronary obstruction, LVOT obstruction, patient-prosthesis mismatch by simulating various clinical scenarios to assess the risk of complications with respect of device design parameters. The computational techniques presented can also be used to guide the development of new devices that allow for control of depth and angle of lacerations for improving procedures such as BASILICA/LAMPOON by testing the device virtually in patient specific geometries for feasibility testing and optimization.

Training databases can also be used to test new device designs such as performing virtual clinical trials. The simulation of the new design can be performed in the training database and its performance pertaining to the desired complication risk assessment can be compared to the current standard used devices. The training could then be used to assess risk for complications in patients outside the training database for the new device design.

### Real-Time Predictive Modelling of Transcatheter Heart Valve Deformations Via Reduced Order Modelling

Transcatheter aortic valve replacement (TAVR) has become an increasingly viable alternative to treat patients with severe aortic stenosis (AS), especially for high surgical risk patients that are unable to undergo traditional surgical valve replacement procedures. Within the TAVR pre-procedural planning pipeline, there is a need for accurate assessment of complications that may occur following the TAVR procedure, such as paravalvular leakage, aortic root rupture, and coronary obstruction. Computational modeling can be a critical tool for visualizing and predicting deployment behavior for TAVR procedures, especially in the context of aortic root rupture.

Computational models are generally created using traditional numerical techniques for solving partial differential equations (PDEs) that govern the fundamental mechanics of the problem. These numerical techniques involve representing the true solution as an approximation, namely as a linear combination of functions involving a finite number of coefficients. The Finite Element (FE) method is the most commonly used technique for predictive modeling of TAVR deployment, where these functions are piecewise polynomials defined over the mesh elements of interest, typically in traditional commercial FE solvers such as Abaqus FEA. Specifically, in TAVR analysis, this process involves modelling the native aortic valve leaflets, aortic root, and calcium deposits, as well as the transcatheter heart valve (THV) stent frame and leaflets. However, due to the intrinsic geometrical and mechanical complexities required to accurately model these components, a very large number of coefficients are required to solve the linear combinations of functions, and thus causes the process to be extremely computationally expensive. Furthermore, coupled interactions of the structural problem with the blood flow only add to complexities of the model, making the process even more computationally expensive.

An exemplary method and system are disclosed that utilizes a Reduced Order Modelling (ROM) framework to rapidly predict the structural deformation of a THV, serving as the first step towards real-time prediction of the entire TAVR deployment procedure. The exemplary method and system allow for a rapid computation of the THV deformations in response to prescribed loads, and is critical in any segregated numerical method, optimization procedures, or in general, iterative schemes where each step requires the solution of the problem several times and under different conditions.

The exemplary system and method require a two phased approach, an offline and online phase, where the computationally expensive simulations are off loaded and performed in the offline stage, followed by a prompt recycling in the online stage for a rapid reduced order solution. In the offline phase, fifteen probing points from the THV model with parameterized loads enforced at each point are used to perform several FE simulations, creating a snapshot library of solutions. Specifically, these fifteen nodes are prescribed in the form of force-pair conditions, where equal and opposite forces between the nodes are prescribed, which leads to net forces pointing radially outwards or inwards from the stent geometry. Different combinations of these force pair are enforced for each simulation, resulting in unique deformations fields that encompass each entry of the snapshot library. The snapshot library is subsequently recycled in the online phase for a new set of applied loads on the same fifteen points via the Proper Orthogonal Decomposition (POD) Galerkin approach. Overall, the framework may be used to significantly reduce the computational costs for simulating the structural deformation of a given THV in response to a defined set of loads.

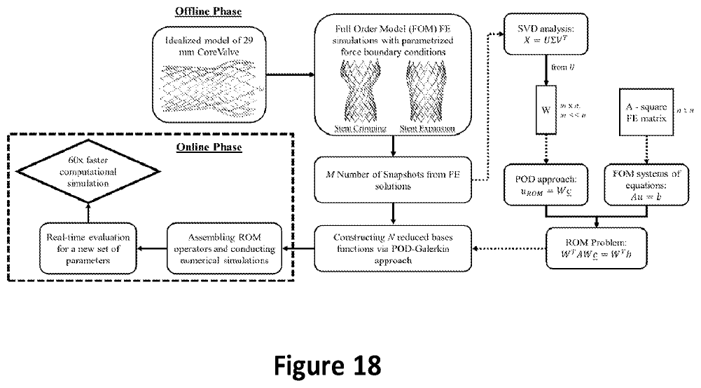

A flowchart describing the exemplary method is shown in FIG. 18, which is primarily composed of an offline and online phase. Briefly, the offline phase begins with a set number of FE simulations with parametrized force boundary conditions at fifteen probing points along the THV stent frame. This is followed by a reduction in dimensionality of the snapshot library via the POD-Galerkin approach, where a uniform selection in the space of parameters is operated, followed by a filtering of the possible redundancy in the snapshot library in a second stage. In the online phase, the reduced basis functions are assembled using the filtered snapshot library, from which the ROM solution is calculated.

Below shows offline phase, idealized model of THV and applied loads. Initially, a 3D geometry of the THV of interest is required to utilize in the exemplary framework. The 3D valve geometry can be reconstructed from reverse-engineering micro-computed tomography (CT) scans of the valve. This process may be done in a computer-aided design software (CAD) such as SolidWorks. In the following examples, a 29 mm Medtronic Evolut R valve stent frame was reconstructed and utilized. It should be noted that the framework can utilize any THV, and all that is needed is the 3D CAD geometry of the valve of interest. The idealized model used in this exemplary framework does not include the pericardium-based leaflets and skirt. This simplification in the model allowed for the exemplary framework to solely focus on the THV stent frame deformations as a first step towards fully capturing TAVR deployment. To perform FE simulations, an adequate mesh of the Evolut valve is created using the open-source platforms Netgen and Mmgtools. In the examples below, the final mesh size was approximately 260K tetrahedral elements. FIG. 18A shows the idealized model of the Evolut R valve stent frame.

To perform FE simulations, the governing mechanics must be defined. In this framework, a linear elastic constitutive law is initially assumed, such that the governing problem for each simulation may read as: find u(x) such that:

$$∇ \cdot σ = F , x ϵΩ . \qquad ( 1 )$$

where σ=λ(∇·u)I+2με is the Cauchy stress tensor, and

$$ε = \frac{1}{2} ( ∇ u + ∇ {u}^{T} )$$

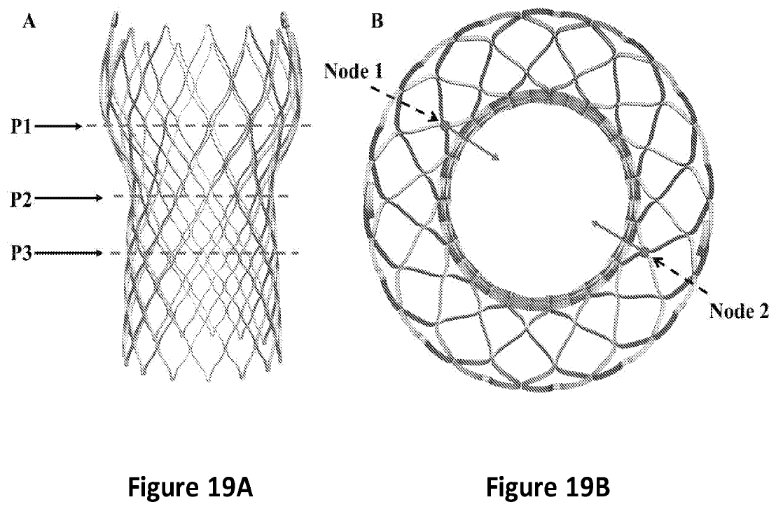

is the strain tensor. I denotes the identity tensor, F is an external volume force, and λ and μ are the Lame' constants. More relevant non-linear constitutive laws such as a hyperelastic (generalized Neo-Hookean model) or superelastic constitutive law may be implemented in this framework as well to accurately describe the governing mechanics of the THV. Using the governing equations, adequate boundary conditions at fifteen probing points are enforced, specifically via “force pairs.” At each of the fifteen points, it was assumed that the normal stress was given, i.e., σ·n(Pᵢ)=dᵢ, for i=1, 2, . . . , 15, where n is the outward normal unit vector. More precisely, all possible pairs among the fifteen points were selected, and dᵢ was defined as a vector oriented along the line connecting the two end points of each pair. These “force pair” boundary conditions idealize the applied loads from the aortic wall and aortic root onto the THV stent frame, as well as mimic the crimping and expansion of the stent frame during the deployment procedure. In addition, it can be shown that any generalized load onto the stent frame can be accurately represented by a sum of force pairs. Utilizing these force pair conditions at the fifteen nodes avoids having to enforce boundary conditions at each of the hundreds of thousands of nodes that compose the stent geometry, thus reducing the complexity of the simulation. This simplification will in turn only enhance the savings in computational costs that are expected from using the ROM framework. A sample force pair between two nodes of the Evolut stent frame is shown in FIG. 19B, and each of these force pairs were defined along the three planes P1, P2, and P3 along the stent frame, as shown in FIG. 19A.

Using these fifteen force pair boundary conditions, 105 FE simulations are performed to form a snapshot library, which essentially acts a training database that can be further utilized in the online phase.

Below shows singular value decomposition of the snapshots. The generated snapshots are a representation of the solution of the governing problem under different boundary conditions. While each snapshot represents a different solution, the level of information each carries about the governing problem may be redundant. As the final goal of this exemplary framework is to construct a set of functions to use for rapid computation in the online phase, an effective filtering of this redundancy is required to create an efficient process. A fundamental tool of linear algebra known as the Singular Value Decomposition (SVD) is employed to filter out this redundancy. The SVD states that given a generic m×n matrix A, it is possible to factorize it into three matrices as follows.

$$A = U \sum {V}^{T} \qquad ( 2 )$$

Here, U is an m×n orthogonal matrix (U^{T}U=I) whose columns are known as the left-singular vectors of A, V is an n×n orthogonal matrix whose columns are the right-singular vectors of A, and Σ is a m×n diagonal matrix whose entries contain the singular values. These singular values from the matrix Σ are ordered in a decreasing fashion, and a rapid decay in the singular values indicates a high redundancy in the dataset that can be filtered such that key features of the snapshot dataset can be captured by a linear combination of those left eigenvectors associated with the largest singular values.

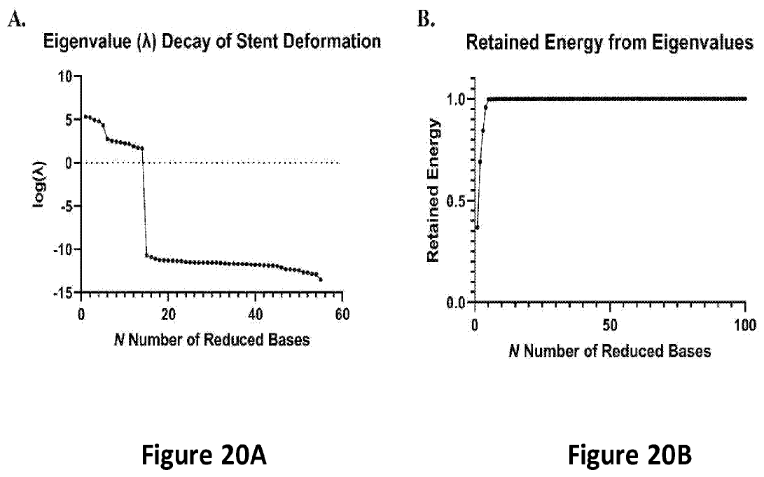

The initial results following the SVD of the generated snapshot library is shown in FIG. 20. A sharp and rapid decline in the singular values is seen after approximately 13 reduced bases, which suggests that the entire full order model (FOM), or the FE problem, can be well approximated by the left eigenvectors associated with the first 13 principal components of the snapshot library. In addition, FIG. 20B displays the retained energy calculated for each reduced basis utilized. The plot plateaus at 13 reduced basis which matches the same number of principal components indicated from FIG. 20A, and this corresponds to 99.99% of the energy captured from the snapshot library. This overall suggests that using only the first 13 principal components is enough to construct the reduced basis functions and calculate the reduced order solution.

Below shows proper orthogonal decomposition-Galerkin approach. Following discretization of the governing problem via the FE method, the FOM from Eq. 1 leads to the solution of a linear system in the following form.

$$Au = b \qquad ( 3 )$$

Here, u is the solution of interest (the resultant displacement), A is the stiffness matrix, and b is a vector that collects the effects of the forcing term and the applied boundary conditions (the force pair conditions). In the POD-Galerkin approach, an approximate solution of Eq. 1 is created, and by plugging this form into Eq. 3, the following reduced order system is created.

$${W}^{T} AWc = {W}^{T} b \qquad ( 4 )$$

In Eq. 4, W is a matrix formed using the filtered left eigenvectors from the SVD analysis. Using Eq. 4 with the vector b incorporating new boundary conditions of interest in the online stage, the “small” vector c can be solved for, from which the final reduced order solution is found. The savings in computational costs become evident when comparing the sizes of A in Eq. 3 to the reduced order matrix W^{T}AW in Eq. 4; the matrix A can be several hundreds of thousands or millions in size, while the W^{T}AW matrix features a size in the range of tens or hundreds of rows. Thus, solving for the system in Eq. 4 requires much less computational costs.

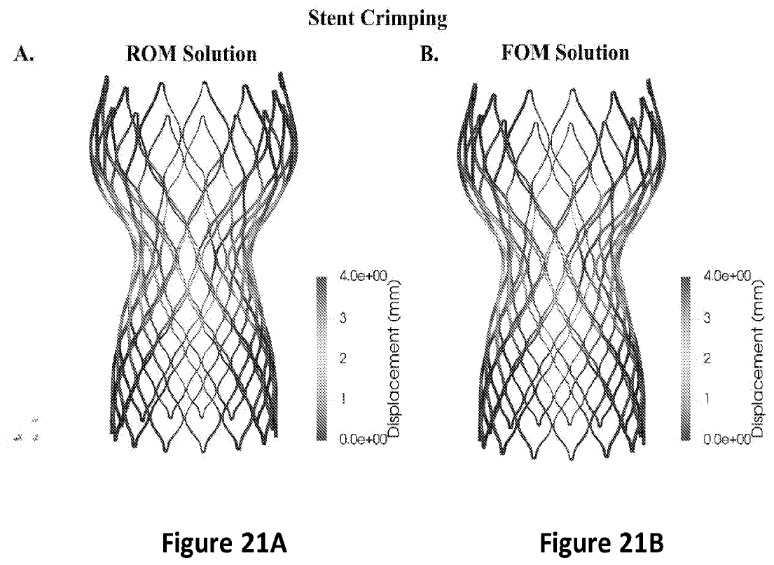

The results using this exemplary framework are shown below. Specifically, in the online phase of the ROM framework, new force boundary conditions were applied and the resulting reduced order solutions were calculated. Using the same force boundary conditions, a traditional FE simulation was also performed to compare the resulting displacement and stresses, as well as computational costs between the ROM and FOM. Two distinct types of simulations were performed: first, simulations where radially inward forces were applied along each of the planes P1, P2, and P3, essentially mimicking stent crimping due to applied loads from the aortic wall, and second, simulations where radially outward forces were applied along each plane, essentially mimicking stent expansion (for example, due to opening and closing of THV leaflets). FIGS. 21A-21B shows the resulting displacements of the stent frame after the radially inward forces were applied. As seen, there is no difference in the ROM solution (FIG. 21A) and the FOM solution (FIG. 21B), which indicates that the exemplary framework provides accurate results as compared to traditional FE simulations.

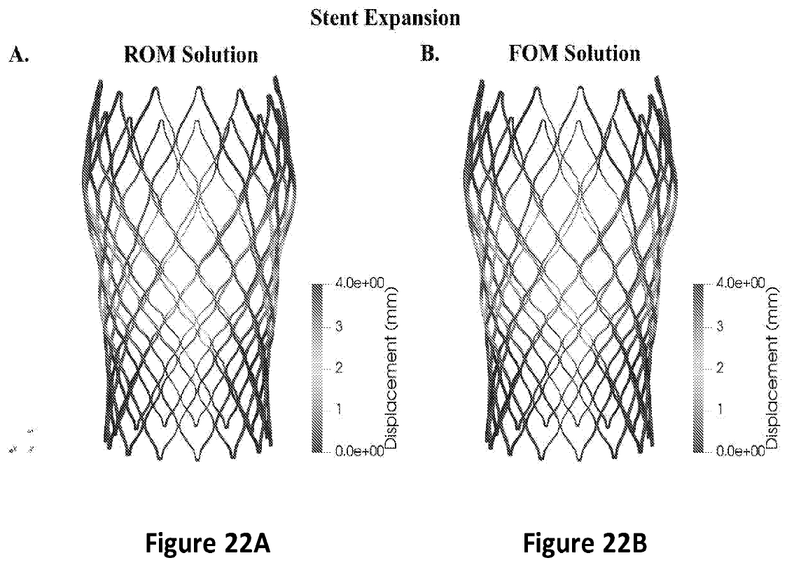

In addition, FIGS. 22A-22B shows the resulting displacements after radially outward forces were applied, which mimic stent expansion. Again, no differences were seen between the ROM solution (FIG. 22A) and the FOM solution (FIG. 22B), highlighting the accuracy of this framework.

The computational details for the ROM and the FOM simulations shown above are summarized in Table 2. FOM simulations required 227,511 degrees of freedom (corresponding to the number of nodes on the THV stent frame), while the ROM simulations utilized only 13 reduced bases to calculate the online solutions. Simulating the FOM FE solution required an average of 122.28 seconds. The average computation time for the ROM offline phase was 2654.39 seconds, while the online phase of the ROM required only an average of 2.14 seconds, a 98.25% decrease in computational time compared to the FOM simulation or approximately 60 times as rapid.

Such a model reduction approach can be developed using common open-source software and libraries. Open-source Python-C++ based finite element libraries such as FEniCS can be used to perform finite element simulations. SVD analysis and the POD-Galerkin process can be performed using common numeric libraries in Python. In addition, the open-source model reduction library RBniCS may be used to capture the entire process. In this case, the entire problem is managed by library, from the offline phase to the model reduction, as well as the speed-up analysis. Table 2 shows summary of computational details for FOM and ROM simulations.

TABLE 2

| | | | | Average Time (s) | |
|---|---|---|---|---|---|
| Mesh | # Computed | # FE | | | ROM |
| Size | Snapshots | DOF | # RB | FE | (Online) |
| 257671 | 105 | 227511 | 12, 13,15 | 122.28 | 2.14 |
| | | | (depending | | |
| | | | on the plane) | | |

Structural analysis of THVs has become an increasingly common area of focus in better understanding the physiological interactions between the valve and the native aortic geometry. Numerous in-silico studies have shown the importance of radial forces applied on the aortic annulus from self-expanding valves, such as the Evolut R, during TAVR deployment, as well as the significance of arterial wall deformation in response to TAVR deployment. The deformation magnitude seen above resemble those found in previous in-silico studies, highlighting the accuracy of the framework. In addition, the uniform deformations of the Evolut valve seen in FIGS. 21A-21B and 22A-22B loosely mimic the deformed states the valve undergoes during physiological deployment. However, conventional FE methods used to model and predict pre-operative TAVR deployment require a large number of degrees of freedom, and in turn, extremely large computation times (up to 24-72 hours). Predicting effects of various valve types and configurations for a single patient may also require multiple simulations, which thus becomes unfeasible for the clinical environment in which rapid and accurate predictive models are required. In the setting of full TAVR deployment, these parameters may include valve positioning within a patient-specific aortic root, different material properties of the valve, or geometrical parameterization of different valve sizes and types. Utilizing the ROM framework allows for a significant reduction in the computational costs as evidenced by Table 2, which primarily occurs due to the number of degrees of freedom utilized in the reduced problem versus the FOM. These savings may be especially practical for simulating TAVR deployment in the clinical environment, as the framework allows for a one-time offline phase followed by the opportunity to use a variety of different parameters that provide accurate and computationally cheap results compared to traditional FE simulations.

An ROM framework based on the POD-Galerkin approach is introduced and applied towards the structural deformation of the 29 mm Medtronic Evolut R valve. Using only fifteen probing points and enforcing “force-pair” loads at each in the offline phase of the framework, a significant decrease in the computation time for simulations that mimic crimping and expansion of the Evolut frame is seen. In addition to the reduced computational costs, the ROM simulations resulted in solutions that were nearly identical to traditional FE methods employed. Further refinements of the framework are underway to rapidly and accurately simulate the TAVR deployment process in its entirety, including addressing limitations such as the use of linear elastic material properties, as well as utilizing more physiologically accurate boundary conditions.

Computational analysis of the post-TAVR hemodynamics is also a critical point of interest for clinicians. Studies have shown that turbulent flow dynamics are created within the aortic root following THV implantation, as well as regions of flow stasis occurring within the aortic root sinus due to patient-specific aortic morphologies. To evaluate these flow features, analysis of the fluid dynamics in response to the THV via CFD is needed. CFD may specifically be used to estimate pressure gradients across the native or transcatheter valve, as well as provide information on blood flow streamlines through the valve, all of which provides clinicians with critical information on the health of the native valve or the performance of the THV. In combination with the structural mechanics of the valve leaflets, this results in a fluid-structure interaction problem that must be solved to provide accurate predictions in patient-specific cases, which dramatically increases the computational costs. ROMs may also play an integral role here in reducing the computational costs associated with flow simulations. Coupled with the ROM for structural simulations of TAVR deployment and with appropriate boundary conditions at the fluid-solid interface, such a combined ROM framework would rapidly provide clinicians with estimations on pressure gradients, regions of flow stasis, and additional pre- and post-operative flow dynamic information for successful TAVR operations in individual patients.

It will be apparent to those skilled in the art that various modifications and variations can be made in the present disclosure without departing from the scope or spirit of the invention. Other aspects of the disclosure will be apparent to those skilled in the art from consideration of the specification and practice of the methods disclosed herein. It is intended that the specification and examples be considered as exemplary only, with a true scope and spirit of the invention being indicated by the following claims.

It should be appreciated that the logical operations described above may be implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

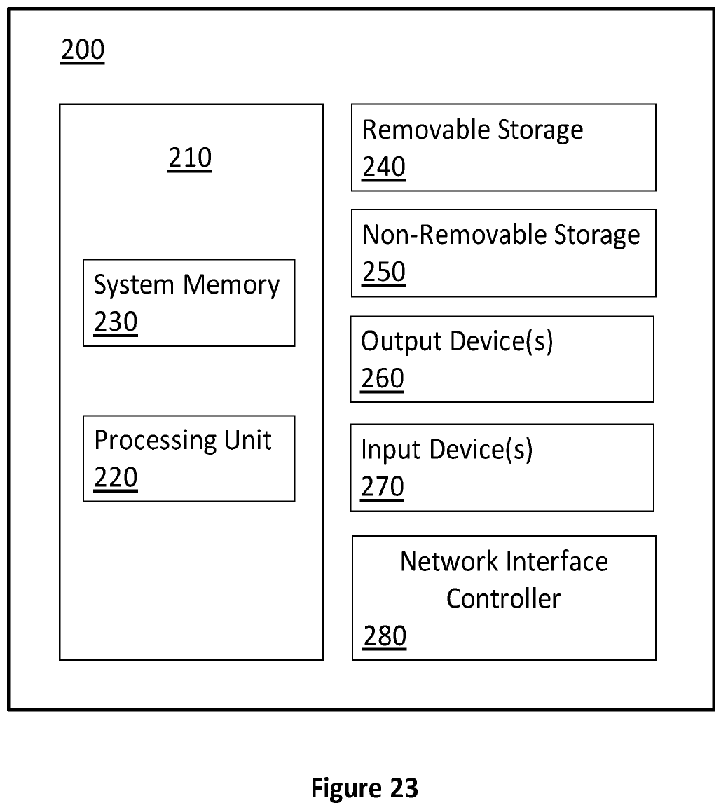

FIG. 23 shows an illustrative computer architecture for a computer system 200 capable of executing the software components that can use the output of the exemplary method described herein. The computer architecture shown in FIG. 23 illustrates an example computer system configuration, and the computer 200 can be utilized to execute any aspects of the components and/or modules presented herein described as executing on the analysis system or any components in communication therewith.

In an aspect, the computing device 200 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an aspect, virtualization software may be employed by the computing device 200 to provide the functionality of a number of servers that is not directly bound to the number of computers in the computing device 200. For example, virtualization software may provide twenty virtual servers on four physical computers. In an aspect, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing device 200 typically includes at least one processing unit 220 and system memory 230. Depending on the exact configuration and type of computing device, system memory 230 may be volatile (such as random-access memory (RAM)), non-volatile (such as read-only memory (ROM), flash memory, etc.), or some combination of the two.

This most basic configuration is illustrated in FIG. 23 by dashed line 210. The processing unit 220 may be a standard programmable processor that performs arithmetic and logic operations necessary for operation of the computing device 200. While only one processing unit 220 is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application specific circuits (ASICs). Thus, while instructions may be discussed as executed by a processor, the instructions may be executed simultaneously, serially, or otherwise executed by one or multiple processors. The computing device 200 may also include a bus or other communication mechanism for communicating information among various components of the computing device 200.

Computing device 200 may have additional features/functionality. For example, computing device 200 may include additional storage such as removable storage 240 and non-removable storage 250 including, but not limited to, magnetic or optical disks or tapes. Computing device 200 may also contain network connection(s) 280 that allow the device to communicate with other devices such as over the communication pathways described herein. The network connection(s) 280 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 200 may also have input device(s) 270 such as keyboards, keypads, switches, dials, mice, track balls, touch screens, voice recognizers, card readers, paper tape readers, or other well-known input devices. Output device(s) 260 such as printers, video monitors, liquid crystal displays (LCDs), touch screen displays, displays, speakers, virtual reality interface etc. may also be included. Interactable interface for real-time user modification of inputs and output visualization can also be implemented to allow the clinician to go through outcomes of various surgical options which would assist in decision making. Real patient imaging such as X-ray angiography can also be fused with the simulation results real-time to offer a more user-friendly environment for clinical practice. The additional devices may be connected to the bus in order to facilitate communication of data among the components of the computing device 200. All these devices are well known in the art and need not be discussed at length here.

The processing unit 220 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 220 for execution. Example tangible, computer-readable media may include, but is not limited to, volatile media, non-volatile media, removable media and non-removable media implemented in any method or technology for storage of information such as computer readable instructions, data structures, program modules or other data. System memory 230, removable storage 240, and non-removable storage 250 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture 200 in order to store and execute the software components presented herein. It also should be appreciated that the computer architecture 200 may include other types of computing devices, including hand-held computers, embedded computer systems, personal digital assistants, and other types of computing devices known to those skilled in the art. It is also contemplated that the computer architecture 200 may not include all of the components shown in FIG. 23, may include other components that are not explicitly shown in FIG. 23, or may utilize an architecture different than that shown in FIG. 23.

In an example implementation, the processing unit 220 may execute program code stored in the system memory 230. For example, the bus may carry data to the system memory 230, from which the processing unit 220 receives and executes instructions. The data received by the system memory 230 may optionally be stored on the removable storage 240 or the non-removable storage 250 before or after execution by the processing unit 220.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Moreover, the various components may be in communication via wireless and/or hardwire or other desirable and available communication means, systems and hardware.

Moreover, various components and modules may be substituted with other modules or components that provide similar functions.

The computer architecture 200 includes software and/or hardware components and modules needed to enable the function of the modeling, simulation, and methods disclosed in the present disclosure. In some aspects, the computer architecture 200 may include artificial intelligence (A.I.) modules or algorithms and/or machine learning (M.L.) modules or algorithms (e.g., stored in the system memory 230, removable storage 240, non-removable storage 250, and/or a cloud database). The A.I. and/or M.L. modules/algorithms may improve the predictive power of the models, simulations, and/or methods disclosed in the present disclosure. For example, by using a deep learning, A.I., and/or M.L. model training including patient information and any relevant input data to the computational model, the predictive power of the computational model may be greatly enhanced. The A.I. and/or M.L. modules/algorithms also help improving sensitivity and specificity of the prediction as the database grows. In some aspects, the computer architecture 200 may include virtual reality (VR), augmented reality (AR) and/or mixed reality display(s), headset(s), glass(es), or any other suitable display device(s) as a part of the output device(s) 260 and/or the input device(s) 270. In some aspects, the display device(s) may be interactive to allow an user to select from options including with or without AR, with or without VR, or fused with real time clinical imaging to help clinician interact and make decisions.

Although example aspects of the present disclosure are explained in some instances in detail herein, it is to be understood that other aspects are contemplated. Accordingly, it is not intended that the present disclosure be limited in its scope to the details of construction and arrangement of components set forth in the following description or illustrated in the drawings. The present disclosure is capable of other aspects and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary aspects include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example e aspects, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure.

Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

What is claimed:

1. A predictive model for classification of tissue rupture risk generated by:
providing a computer aided design (CAD) model suitable for simulating an expandable transcatheter heart valve; and
computing stress, strain, and/or displacement at the tissue as a function of expansion of the expandable transcatheter heart valve;
wherein the computed stress, strain, and/or displacement at the tissue enables determination of low, moderate or high tissue rupture risk as a function of the expansion at time of the expandable transcatheter heart valve deployment into a patient.

2. The predictive model of claim 1, wherein the tissue comprises tissue of an aortic root.

3. The predictive model of claim 1, wherein the CAD model is built based on a CT scan, micro-CT scan, MRI scan, and/or a CAD geometry model of the expandable transcatheter heart valve.

4. The predictive model of claim 1 is further generated by altering a depth, angle, or position of the expandable transcatheter heart valve to reduce a computed risk of tissue rupture.

5. The predictive model of claim 1, wherein the computed stress, strain, and/or displacement are due to calcium protrusion, calcium nodule, or caused by the curvature contours of a specific anatomical structure.

6. The predictive model of claim 1, wherein the stress, strain, and/or displacement is computed based on reduced order modeling.

7. The predictive model of claim 6, wherein the reduced order modeling is developed based on machine learning framework, deep learning framework, and/or artificial neural networks framework.

8. A method of generating a predictive model for classification of tissue rupture risk comprising:
providing a computer aided design (CAD) model suitable for simulating an expandable transcatheter heart valve; and
computing stress, strain, and/or displacement at the tissue as a function of expansion of the expandable transcatheter heart valve;
wherein the computed stress, strain, and/or displacement at the tissue enables determination of low, moderate or high tissue rupture risk as a function of the expansion at time of the expandable transcatheter heart valve deployment into a patient.

9. The method of claim 8, comprising building the CAD model based on a CT scan, micro-CT scan, MRI scan, and/or a CAD geometry model of the expandable transcatheter heart valve.

10. The method of claim 8, further comprising:
generating illustrations of the stress, strain, and/or displacement at the tissue as a function of the expansion before a transcatheter aortic valve replacement procedure; and
determining an optimum expansion for the patient thereby reducing the tissue rupture risk to the patient.

11. The method of claim 8, comprising computing the stress, strain, and/or displacement based on reduced order modeling.

12. A patient-specific preoperative model generated by:
obtaining patient CT scans;
generating an anatomical model; and
simulating THV deployment at multiple depths and angles of THV, positions or depth of lacerations for BASILICA/LAMPOON, balloon filling volumes and pressures, tissue modifications in the patient anatomy model to determine optimal surgical values for each variable.

13. The patient specific preoperative model of claim 12, wherein generating the anatomical model comprises modeling of aortic root, native aortic or mitral valve, left ventricle, left atrium, calcium, a bioprosthetic valve or stent, and leaflets.

14. The patient specific preoperative model of claim 12, wherein the patient has no previous interventions or alteration of heart anatomy.

15. The patient specific preoperative model of claim 12, wherein the patient anatomy comprises a previously implanted stented bioprosthetic heart valve in need of fracture and replacement.

16. The patient specific preoperative model of claim 15, further comprising measurement of metrics such as coronary flow velocities, pressure gradients across heart valves and how it changes with SHV fracture and/or laceration depth, angle, THV valve sizing for further procedural optimization of transcatheter heart valve simulation inside stented bioprosthetic heart valve or native heart valve with tissue modifications.

17. A method of preoperatively evaluating the success of a transcatheter heart valve replacement procedure in a patient comprising:
obtaining patient CT scans;
generating a patient anatomy model; and
simulating THV deployment depth, angle of THV, position or depth of laceration for BASILICA/LAMPOON, balloon volume and pressure, tissue modifications in the patient anatomy model to determine optimal surgical values for each variable.

18. The method of claim 17, wherein generating the patient anatomy model comprises modeling of aortic root, native aortic or mitral valve, left ventricle, left atrium, calcium, a bioprosthetic valve or stent, and leaflets.

19. The method of claim 17, wherein the patient has no previous interventions or alteration of heart anatomy.

20. The method of claim 17, wherein the patient anatomy model comprises a previously implanted stented bioprosthetic heart valve in need of fracture and replacement.

21. The method of claim 20, wherein the patient anatomy model further comprises measurement of metrics such as coronary flow velocities, pressure gradients and how it changes with SHV fracture and/or laceration depth, angle, THV valve sizing for further procedural optimization of transcatheter heart valve simulation inside stented bioprosthetic heart valve or native heart valve with tissue modifications.

22. A method for predictive modeling of transcatheter heart valve deformation using reduced order modeling, comprising:
obtaining a library of solutions of selective nodes of a transcatheter heart valve model with a first set of force boundary conditions applied to the selective nodes via finite element simulations; and
predicting deformation of the transcatheter heart valve under a second set of force boundary conditions on the selective nodes via a reduced order model, wherein the second set of force boundary conditions are different from the first set of force boundary conditions.

23. The method of claim 22, comprising choosing the selective nodes from hundreds or thousands of nodes that compose stent geometry of the transcatheter heart valve.

24. The method of claim 22, comprising predicting the deformation of the transcatheter heart valve under the second set of force boundary conditions via a Proper Orthogonal Decomposition (POD) Galerkin approach.

25. The method of claim 22, wherein the selective nodes comprise fewer than 20 nodes.

26. The method of claim 22, wherein the deformation of the transcatheter heart valve is at time of deployment of the transcatheter heart valve into a patient specific geometry.
